(19) United States
(12) Patent Application Publication
(10) Pub. No.: US 2015/0216601 A1
(43) Pub. Date: Aug. 6, 2015

(54) PROTECTIVE SHEATH POSITIONING ARRANGEMENT AND METHOD, AND MINIATURE FIBER LOCK CONNECTOR FOR USE THEREWITH

(71) Applicant: Joe Denton Brown, Panama City Beach, FL (US)

(72) Inventor: Joe Denton Brown, Panama City Beach, FL (US)

(21) Appl. No.: 14/598,799

(22) Filed: Jan. 16, 2015

Related U.S. Application Data
(63) Continuation-in-part of application No. 14/218,407, filed on Mar. 18, 2014.
Application No. 14/218,407 is a continuation-in-part of application No. 14/520,551, filed on Oct. 22, 2014.
(60) Provisional application No. 61/928,047, filed on Jan. 16, 2014.
(60) Provisional application No. 61/787,599, filed on Mar. 15, 2013.
(60) Provisional application No. 61/819,900, filed on May 6, 2013.
(60) Provisional application No. 61/824,755, filed on May 17, 2013.
(60) Provisional application No. 61/894,393, filed on Oct. 22, 2013.

(51) Int. Cl. A61B 19/02; A61B 1/00; A61B 1/307; A61B 18/24; A61B 1/018

(52) U.S. Cl. CPC A61B 19/02; A61B 18/24; A61B 1/018; A61B 1/307; A61B 1/0014; A61B 2018/2205

(57) ABSTRACT

A protective sheath of the type used to protect a scope during insertion of a fiber into the scope includes marks on an outer diameter that enable positioning of the sheath relative to a scope with the need for a locking connector. The sheath can be held in the correct position by a physician's choice of leakage prevention device. A simplified miniature connector may be used to hold the fiber in position relative to the sheath.

This application claims the benefit of U.S. Provisional Patent Appl. Ser. No. 61/928,047, filed Jan. 16, 2014, and incorporated herein by reference.

This application is a continuation-in-part of U.S. patent appl. Ser. No. 14/218,907, filed Mar. 15, 2014, which claims the benefit of U.S. Provisional Patent Appl. Ser. Nos. 61/787,599, filed Mar. 15, 2013, 61/819,900, filed May 6, 2013, and 61/824,755, filed May 17, 2013, each of which is incorporated herein by reference.

This application is also a continuation-in-part of U.S. patent appl. Ser. No. 14/520,551, filed Oct. 22, 2014, which claims the benefit of U.S. Provisional Patent Appl. Ser. No. 61/894,393, filed Oct. 22, 2013, each of which is incorporated herein by reference.

### BACKGROUND OF THE INVENTION

1. Field of the Invention

This invention generally relates to an apparatus and method for delivering therapeutic light to a tissue, and in particular to an optical fiber arrangement in which a protective sheath is placed over the entire length of the fiber prior to insertion into an endoscope that guides the fiber to a treatment site. The protective sheath prevents mechanical damage to working channel of the endoscope during insertion of the fiber, insulates the fiber from surrounding cooling fluids, and may serve as an indicator of overheating that enables early detection of excess heating or burning of tissues or equipment at the treatment site.

The present invention provides improvements to the apparatus and methods disclosed in parent U.S. patent appl. Ser. No. 14/218,907. The improvements involve placement of a mark on an externally visible section of the sheath to give a physician an externally visible indication of the correct position of the sheath relative to a scope or introducer, use of a floating sheath and a leak prevention device to hold the floating sheath in place, and an improved arrangement for locking the fiber relative to the free floating sheath that replaces the previously disclosed locking connector or “”FiberLok™“” device.

2. Description of Related Art

The inventor's U.S. patent appl. Ser. No. 13/127,911, filed May 5, 2011 (based on PCT Appl. No. PCT/US2009/006021) and the inventor's copending PCT Appl. No. PCT/US2009/006021, filed Nov. 6, 2009, disclosed protective sheaths that surround a laser delivery fiber during insertion of the fiber into a scope or introducer. One purpose of these sheaths was to allow the advancement of the relatively sharp-edged laser fiber tip through a ureteroscope without damaging the inner wall of the scope's working channel.

The initial design of the protective sheaths provided for a free-floating sheath with a free floating fiber inside the sheath. According to one commercial implementation used for ureteroscopic applications, a Touhy-Borst (TB) connector was locked onto the Luer connector on the scope, and the TB was tightened onto the reinforced section of the sheath to lock the sheath in the desired position relative to the scope. However, the initial design had the drawback in that, during a surgical procedure, the physician did not know where the fiber was relative to the sheath or where the sheath was relative to the scope.

As disclosed in parent U.S. patent appl. Ser. No. 14/218,907, this drawback was addressed by enabling the physician to lock the sheath in the optimal position relative to the scope with a Luer lock connector. Marks on the fiber allowed the physician to easily and precisely position the fiber tip just inside the sheath during fiber/sheath assembly insertion into the scope to prevent damage to the scope.

In addition, to address the problem of a much larger than expected variation in the length of the Storz scopes used in the field (a 5 mm range of variation in scope lengths instead of 1 mm), an adjustable Luer lock connector (or “FiberLok™”) was designed. However, this arrangement still resulted in drawbacks, including an overall length and weight of the device that was greater than desired, and limitations in the variety of scopes to which the arrangement could be applied.

### SUMMARY OF THE INVENTION

It is accordingly a first objective of the invention to provide various improvements to a positioning arrangement for a protective sheath that surrounds a laser delivery fiber during insertion of the fiber into a scope or introducer.

It is a second objective of the invention to provide a low cost, easier-to-use alternative to the previously proposed fiber positioning arrangements that functions with a wide variety of scopes and leak prevention device.

It is a third objective of the invention to provide an alternative to the previously proposed fiber positioning arrangement that is able to accommodate variations in lengths between various scope manufacturers as well as the variation experienced in a single manufacturer's scope length.

It is a fourth objective of the invention to provide an improved method of positioning a laser delivery fiber surrounded by a protective sheath.

These objectives of the invention are achieved, in accordance with a preferred embodiment of the invention, by placing a mark on the outer diameter of the sheath assembly outside of the scope that allows the physician to confidently place the floating version of the sheath in the correct position relative to the scope and leak prevention device. The floating sheath is held in place by the physician's choice of leak prevention devices. A plurality of marks may then be used to accommodate various scopes and leak prevention devices. Each mark may be coded for a particular ureteroscope and leak prevention type, and each scope may have a distinctive primary mark plus a series of smaller markings at intervals to cover the range of positions possible utilizing that scope. The primary mark gets the sheath in the approximate position, after which the physician adjusts the final sheath position under visual control. This technique allows the use of a single set of marks on the fiber to accommodate all scopes.

Alternatively, according to a second that accommodates the variation in length between various scope manufacturers and the variation experienced in a single manufacturer's scope length, a single mark may be used on the sheath at a position exterior to the scope, but additional marks on the distal end of the sheath indicate where the physician should cut the sheath to adjust the length of the sheath to each scope. The ability of this embodiments to accommodate greater variations or manufacturing tolerances in scope length, however, also results in the drawback that the marks on the fiber must also be coded to ensure that the physician uses the correct marking on the fiber to function with the cut sheath length.

Although the positioning apparatus and method of the invention are described in connection with ureteroscopic applications, it will be appreciated that the apparatus and method may be applied to other types of endoscope or introducer and therefore are not limited to ureteroscopic applications.

### BRIEF DESCRIPTION OF THE DRAWINGS

FIG. 1 is a close-up side view of a fiber positioning arrangement that enables position of the fiber tip relative to a protective sheath before insertion into a scope in accordance with the principles of a preferred embodiment of the invention.

FIG. 2 is a side view of a bare laser fiber with an SMA905 connector, and to which marks may be applied for positioning the fiber relative to the sheath and also for recleaning to predetermined lengths.

FIG. 3 is a side view of a laser fiber of the type shown in FIG. 2 and having marks for positioning the fiber relative to the sheath and marks on near the tip to aid re-cleaving of the fiber.

FIG. 4 is a side view of a sheath design with marks on the sheath to aid positioning of the sheath relative to a particular scope according to a preferred embodiment of the invention.

FIG. 5 is a side view of a positioning arrangement that includes a leak prevention device and an adjustable Luer lock connector with a locking insert according to a preferred embodiment of the invention.

FIG. 6 is a side view showing details of a locking arrangement suitable for use in the connection with the embodiment of FIG. 4 and that replaces the Luer lock of FIGS. 2-3 by a miniature locking device to allow the fiber to be positioned correctly relative to a sheath using markings on the fiber, according to a preferred embodiment of the invention.

FIG. 7 shows details of the miniature locking device of FIG. 6.

### DETAILED DESCRIPTION OF THE PREFERRED EMBODIMENTS

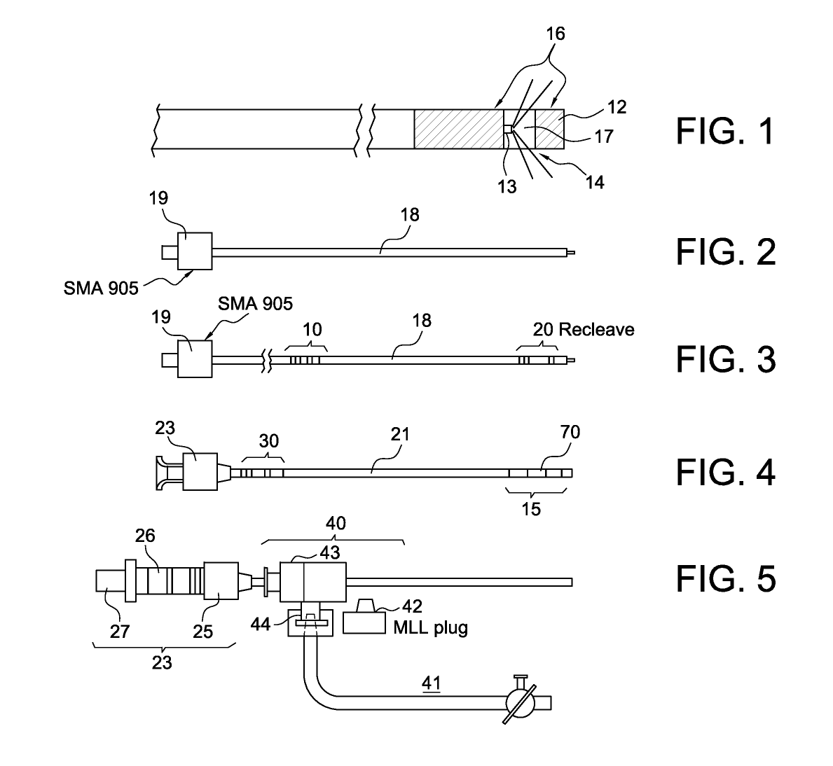

FIG. 1 is close up side view of an arrangement that facilitates positioning of a fiber tip 13 relative to a protective sheath 12 prior to insertion into the scope. The fiber tip positioning arrangement shown in FIG. 1 involves printing black marks 16 on an outer diameter of the tip of a polyimide sleeve leaving a small window 17 to indicate when the fiber tip 13 is in the correct position. The aiming beam (indicated by arrows 14) at the end of the fiber will be muted by the marks, and then will appear to suddenly brighten at the desired tip location. If the fiber goes too far, i.e., past the window 17, then the beam will become muted again. This will allow the physician to more rapidly adjust and verify tip position, even after re-cleaving the fiber. One advantage of this design is that it can be used with physician's usual choice of leak prevention device. The sheath 12 can also be marked at a position at the entrance to the leak prevention device to indicate desired sheath position relative to the scope tip.

FIG. 3 shows a fiber 18 with an SMA connector 19 and markings 10 for positioning the fiber relative to the connector, as well as markings 20 at a distal end to indicate standard or predetermined fiber lengths for recleaning the fiber. These features are disclosed in the inventor's earlier applications. FIG. 2 shows fiber 18 with SMA connector 19 but no markings for comparison.

FIG. 4 shows a sheath and fiber positioning connector arrangement 23 and sheath 21 marked in accordance with an embodiment of the present invention, with an enlarged view of the sheath and fiber positioning arrangement being included in FIG. 5. As shown in FIG. 4, one or more marks 30 are included on the outer diameter of the sheath assembly outside of the scope that allows the physician to confidently place the floating version of the sheath in the correct position relative to the scope and leak prevention device. The floating sheath is held in place by the physician's choice of leak prevention devices.

As shown in FIG. 4, the marks 30 on the sheath 21 aid in sheath positioning relative to a particular scope, while marks 10 on the fiber 18 as shown in FIG. 3 aid in positioning of the fiber. Marks 15 near the tip of the sheath allow physician to tailor the length to the individual scope and leak prevention device combination, while marks 20 on the fiber of FIG. 3 have the same purpose with respect to the length of the fiber.

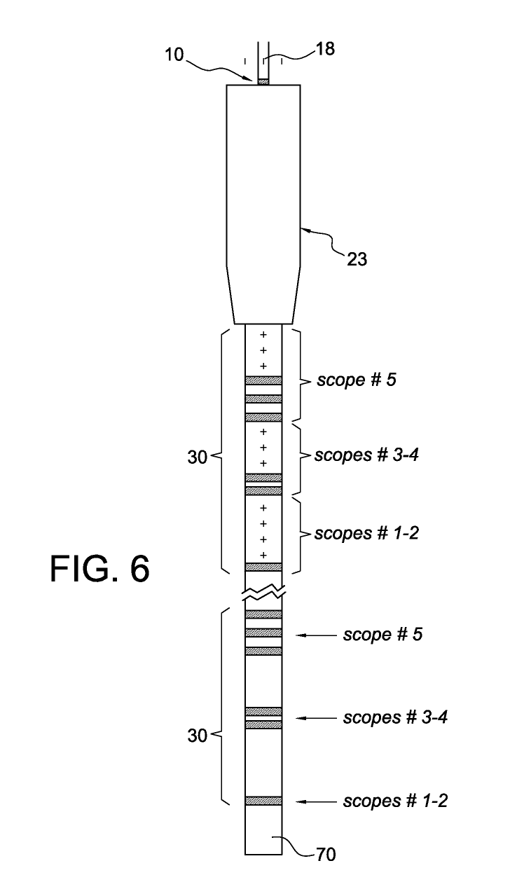

As best shown in FIG. 6, a plurality of marks may be used to accommodate various scopes and leak prevention devices. Each mark may be coded for a particular ureteroscope and leak prevention type. Each scope may have a distinctive primary mark plus a series of smaller markings at intervals to cover the range of positions possible utilizing that scope. The primary mark gets the sheath 21 in the approximate position, and then the physician adjusts the final sheath position under visual control. This technique allows the use of a single set of marks on the fiber to accommodate all scopes.

Alternatively, as also shown in FIG. 6, to accommodate the variation in length between various scope manufacturers and the variation experienced in a single manufacturer's scope length, marks 15 (which are in addition to marks 30) on the distal end 70 of the sheath may be added to indicate where the physician should cut the sheath to adjust the length of the sheath to each scope. This technique, however, has the drawback that the marks on the fiber must also be coded to ensure that the physician uses the correct marking on the fiber to function with the cut sheath length.

The distal end 70 of the fiber may be provided with a material in the form of a coating or ingredient, such as a phosphor, that provides a signal that allows rapid detection and warning that the physician has pulled the laser fiber into the sheath or that the fiber has broken in the working channel. The sheath and/or fiber positioning connector arrangement 23 may optionally take the form illustrated in FIGS. 5-7.

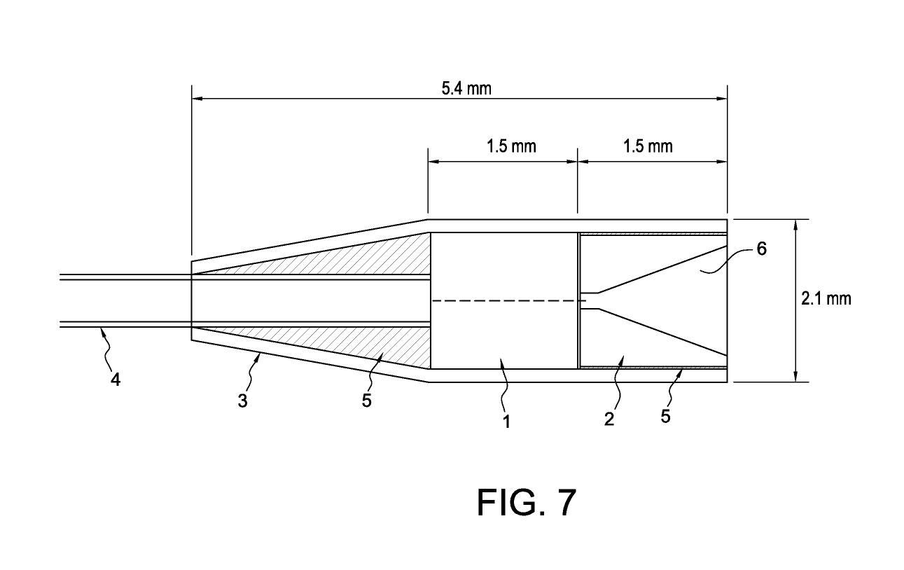

To allow the physician to position the fiber correctly relative to the free-floating sheath with markings, as described above, a new design was needed to replace the larger, more expensive device described in parent U.S. patent appl. Ser. No. 14/218,907 (and available from Optical Integrity, Inc. under the trademark FiberLok™). Use of a miniature locking device 23, as shown in FIG. 4, allows the fiber to be positioned correctly relative to the sheath using markings on the fiber, as previously disclosed. Since there is no Luer lock connector to attach the miniature fiber locking device 23 to, a completely new design was needed. As shown in more detail in FIGS. 6 and 7, the fiber locking device of this embodiment includes a small silicone insert or seal 1 sandwiched between the existing reinforced section 4 of the sheath 21 and a cylinder 2 with a conical opening 6 that guides the optical fiber (or other tool) to the pre-pierced center of the silicone insert. These components are held in place by an adhesive 5 applied to a thin outer tube 3.

According to this design, as shown in FIG. 7, the outer plastic sleeve 3 is bonded to a silicon seal 1 and a reinforced section 4 of sheath 21 by adhesive 5. The cylindrical plastic insert 2 with the conical opening 6 ensures that the fiber enters a pre-pierced portion of the silicon seal. The seal 1 holds the sheath during insertion into the scope, but still allows pre-positioning of sheath based on the markings on the sheath

This design is significantly smaller and cheaper than the previous fiber locking device. The previous fiber locking device is 15.5 mm long by 13 mm in diameter and costs about $2.80 each. In contrast, the novel design has a silicone insert that is about 1.5 mm in diameter and 0.75 mm thick; the overall miniature fiber lock of the preferred embodiment is approximately 2 mm in diameter and about 6 mm long.

The silicone insert 1 is pierced with a needle to provide a path for the laser fiber to follow; this allows a slightly easier initial insertion force to facilitate manufacturing and ensures more reproducible resistance to fiber movement. The previous fiber locking connector design was so large that it may have interfered with the physician's technique of gripping the scope and manipulating the fiber, while the new design has minimal impact on the physician's technique. This design is very light weight and low profile to allow the physician to manipulate the fiber in his accustomed manner.

In addition, the large diameter of the fiber locking/Luer lock device disclosed in parent U.S. patent appl. Ser. No. 14/218,907 prevented it from being inserted into the coiled hoop used to package the fiber assembly. The small diameter of the miniature design of the presently illustrated preferred embodiment allows the manufacturer to pre-position the sheath on correct position on the fiber, which allows the physician to pull the assembly from the packaging hoop and insert it directly into the leak prevention device on the working channel of the scope without any adjustment. Previously, the sheath had to be slid about 2 meters up to the SMA connector to allow packaging into the hoop. This required the physician to pull about two meters of fiber from the hoop watching for the marks on the fiber; then when the marks appeared, he would unlock the Luer lock/fiber locking assembly from the hoop and remove the adjusted assembly from the hoop and insert it into the scope. In addition, during the packaging process, a Teflon sleeve was needed to cover the sharp end of the polished fiber during insertion through the hoop, which was then recovered after insertion and reused for the next fiber insertion. The preferred miniature fiber locking arrangement obviates the need for a Teflon sleeve since the sheath is already positioned to cover the fiber tip and facilitate insertion into the hoop (much the same as it is designed to protect the working channel of the flexed scope).

FIG. 5 illustrates a design that comprises both a leak prevention device 40 and an adjustable Luer lock connector 25 with a fiber locking insert 27 that may correspond to the one shown in FIG. 7. The marks 26 demonstrate an optional concept for measuring position that would allow a physician to preset the sheath position for his favorite scope which he knows is at the third mark. The leak prevention device can be used with a plug 42 or irrigation can be provided through an alternative sidearm with valve 41 that connects to the main body of the one way valve 43.

I claim:

1. A protective sheath for an optical fiber used in surgical laser procedures, wherein the protective sheath includes marking on an outside diameter of the sheath to allow a physician to position the sheath relative to a scope.

2. A protective sheath as claimed in claim 1, wherein the sheath is a floating sheath that is held in place following positioning by a leak prevention device.

3. A protective sheath as claimed in claim 1, wherein a plurality of said marks are provided, said marks being coded to a particular scope and/or leak prevention device type.

4. A protective sheath as claimed in claim 1, wherein the at least one mark includes a primary mark and a series of smaller marks, the primary marks indicating an approximate position of the sheath, and the smaller marks enabling finer positioning of the sheath by the physician.

5. A protective sheath as claimed in claim 1, further comprising additional marks at a distal end of the sheath to enable cutting of the sheath to adjust its length.

6. A protective sheath as claimed in claim 1, wherein the at least one mark include marks provide at a distal end of the fiber and a window is included in the sheath to indicate when the fiber tip is in a correct position relative to a tip of the scope, the black marks muting an aiming beam until the tip is in the correct position, at which time the aiming beam will appear to suddenly brighten, and will become muted again if the fiber tip travels beyond the correct position.

7. A protective sheath as claimed in claim 6, wherein the sheath is also marked at a position near the entrance of a leak prevention device to indicate a desired sheath position relative to the scope tip.

8. A protective sheath as claimed in claim 1, wherein the scope is a ureteroscope.

9. A protective sheath as claimed in claim 1, wherein the fiber includes an SMA connector and additional marking for positioning the fiber relative to the sheath.

10. A protective sheath as claimed in claim 9, wherein the fiber further includes markings at a distal end of the fiber for recleaning the fiber.

11. A protective sheath as claimed in claim 1, further comprising a coating or material at a distal end of the sheath for providing a signal that allows rapid detection and warning that the fiber has been pulled into the sheath during lasing, or that the fiber has broken in the working channel.

12. A protective sheath as claimed in claim 11, wherein the coating or material is a phosphor.

13. Surgical laser equipment including the sheath of any preceding claim and a miniature fiber locking connector that eliminates the need for a Luer connector, the miniature locking connector including a small silicone insert sandwiched between an existing reinforced section of the sheath and a cylinder with a conical opening that guides the fiber or other tool to a pre-pierced center of the silicone insert, the silicon insert and sheath being held in place by an adhesive and thin outer tube.

14. A miniature fiber locking connector that eliminates the need for a Luer connector, comprising: a small silicone insert sandwiched between an existing reinforced section of a fiber-protective sheath and a cylinder with a conical opening that guides the fiber or other tool to a pre-pierced center of the silicone insert, the silicon insert and sheath being held in place by an adhesive and thin outer tube.

15. A method of positioning a fiber relative to a scope,
comprising the steps of:
a user positioning a free-floating sheath relative to a scope by using marks on an exterior diameter of the sheath; and
holding the sheath in a correct position relative to the scope by a leak prevention device.

16. A method as claimed in claim 15, wherein the step of using the marks comprises the steps of using a primary mark on the sheath to place the sheath in an approximate position and smaller marks on the sheath to provide finer alignment under visual control.

17. A method as claimed in claim 15, further comprising the step of observing an aiming beam that is muted by the marks, and determining that a tip of the fiber is correctly positioned when the aiming beam brightens as a result of the tip being positioned in an area of a window between the marks.

18. A method of adjusting a length of a sheath, comprising the step of using re-cleaving markings on the sheath as a guide for cutting of the sheath without locking the sheath with respect to a locking connector.
